(19) United States
(12) Patent Application Publication
(10) Pub. No.: US 2010/0255105 A1
(43) Pub. Date: Oct. 7, 2010

(54) EXTENDED RELEASE PHARMACEUTICAL COMPOSITION COMPRISING METOPROLOL SUCCINATE

(75) Inventors: CHITRA SHAH, Mumbai (IN); FRANCISCO GUAL PUJOL, Barcelona (ES); JOAQUÍN AMELA-NAVARRO, Barcelona (ES); JOSÉ-LUIS RUIZ-CÓRDOBA, Barcelona (ES)

Correspondence Address: BERENBAUM WEINSHIENK PC, 370 17TH STREET, SUITE 4800, DENVER, CO, 80202, US

(73) Assignee: ZAKLADY FARMACEUTYCZNE POLPHARMA S.A., Starograd Gdanski (PL)

(21) Appl. No.: 12/751,848

(22) Filed: Mar. 31, 2010

(30) Foreign Application Priority Data
Apr. 3, 2009 (EP) EP09157343.6
May 4, 2009 (EP) EP09159296.4

(51) Int. Cl. A61K 9/14; A61K 31/135; A61P 9/12

(52) U.S. Cl. 424/489; 514/651

(57) ABSTRACT

An extended release pharmaceutical composition comprising metoprolol succinate and at least two pharmaceutically acceptable excipients, wherein the first pharmaceutically acceptable excipient is an extended release agent; the second pharmaceutically acceptable excipient is selected from a binder, a diluent and mixtures thereof; and metoprolol succinate is in a crystalline form having a D50 ranging from 5 to 16 microns and a D90 below 50 microns.

### FIELD OF THE INVENTION

The present invention relates to extended release pharmaceutical compositions comprising metoprolol succinate having a small particle size.

### BACKGROUND OF THE INVENTION

Metoprolol is a selective β1 receptor blocker used in the treatment of several diseases of the cardiovascular system, especially hypertension. Metoprolol is marketed in the form of succinate salt.

European patent application EP 293347-A describes, for the first time, metoprolol succinate and an oral pharmaceutical composition which comprises a core containing a therapeutically active compound coated with a layer comprising a) 10 to 85% by weight of an anionic polymer soluble at a pH above 5.5, and b) 15 to 90% by weight of a water-insoluble polymer selected from quaternary ammonium-substituted acrylic polymers.

WO2008012346 A discloses coated granules consisting of granules having a particle size ranging from 0.2 to 2 mm, friability lower than or equal to 1% and comprising metoprolol succinate as active ingredient in an amount ranging from 10 to 75% by weight of the granule and at least one binder selected from microcrystalline cellulose and methylcellulose, coated with a film-former coating agent. These granules are used to prepare extended-release pharmaceutical compositions. Particle size of the granules ranging from 0.2 to 1 mm are claimed, but no information about the particle size of the metoprolol succinate used to prepare the granules is provided.

EP0220143 A discloses controlled release preparations containing a salt of metoprolol characterized in that the preparation contains a number of beads comprising a salt of metoprolol as the main soluble component and that said beads are coated with a polymeric membrane containing derivatives of cellulose without protolysable groups and whereby at least 75% of the dose of metoprolol is released within 20 hours virtually independent of the pH in the interval 1-8. The size of the beads in the range of 0.25-2 mm is claimed, but no information about the particle size of the salt of metoprolol used to prepare the beads is provided.

US2005008701 A1 discloses controlled release pellets of metoprolol having an inert core, a drug layer optionally comprising a binder and a controlled release coating surrounding that drug layer. The diameter of the inner core is an important feature of the invention. Such pellets are used to prepare extended-release pharmaceutical compositions. Pellets having a diameter of the inert core of less than 30 mesh are claimed, but no information about the particle size of the metoprolol used to prepare pellets is provided.

EP0311582 A discloses controlled release preparations for administration once daily and containing a combination of metoprolol and a poorly water soluble calcium channel blocking agent of the dihydropyridine type, wherein metoprolol is included in the form of small beads containing as the main soluble component a salt of metoprolol coated with a water-insoluble polymeric membrane and the dihydropyridine is dispersed in a non-ionic solubilizer and whereby both the dispersed dihydropyridine and the beads containing metoprolol are incorporated into a matrix forming a swelling gel in contact with water. No information about the particle size of the metoprolol used to prepare the beads that are incorporated into a matrix forming a swelling gel in contact with water is provided.

WO2004069234 A discloses pharmaceutical compositions comprising a matrix material having metoprolol, or a pharmaceutically acceptable salt thereof, dispersed therein, the dispersion of the metoprolol or pharmaceutically acceptable salt thereof within the matrix material being effective to delay the release profile on administration of the pharmaceutical composition, the tablet being provided with a substantially water-insoluble polymeric coating effective further to delay the release profile on administration of the pharmaceutical composition. No information about the particle size of the metoprolol succinate used to prepare the matrix material is provided.

Time Release Technology also known as sustained-release, extended-release, time-release or timed-release, controlled-release, or continuous-release pills or tablets or capsules formulated to dissolve slowly and release a drug over time. The advantages of sustained-release tablets or capsules are that they can often be taken less frequently than instant-release formulations of the same drug, and that they maintain steadier levels of the drug in the bloodstream.

One of the major problems in the development of extended release pharmaceutical compositions comprising metoprolol succinate is its high solubility, being freely soluble in water. The solubility in water at 37° C. is 276 mg/mL (see Ragnarsson et al., International Journal of Pharmaceutics, 1992, vol. 79, n° 2-3, pp. 223-232).

For example, the authors of Remington: The Science and Practice of Pharmacy, Lippincott Willians & Wilkins, 20th Edition, page 907, state that “in general, extremes in the aqueous solubility of a drug are undesirable for a formulation into a controlled-release product. A drug with very low solubility and slow dissolution rate will exhibit dissolution-limited absorption and yield an inherently sustained blood level.” Also the authors further hold that “for a drug with a very high solubility and rapid dissolution rate, it often is quite difficult to decrease its dissolution rate and slow its absorption. Preparing a slightly soluble form of a drug with normally high solubility is, however, one possible method for preparing extended release dosage forms.”

### SUMMARY OF THE INVENTION

The problem to be solved by the present invention is to provide an alternative extended release pharmaceutical composition comprising metoprolol succinate which has a release profile close to zero order, and adequate pharmacokinetic parameters, and at the same time being easy to manufacture and with suitable pharmacotechnical parameters.

The first aspect of the present invention is an extended release pharmaceutical composition comprising metoprolol succinate and at least two pharmaceutically acceptable excipients, wherein one pharmaceutically acceptable excipient is an extended release agent; the second pharmaceutically acceptable excipient is selected from a binder, a diluent and mixtures thereof; and metoprolol succinate is in a crystalline form having a D50 ranging from 5 to 16 microns and a D90 below 50 microns. The inventors have surprisingly found that the behavior of such extended release pharmaceutical compositions comprising metoprolol succinate is just the opposite from that expected by a person skilled in the art. According to common knowledge, the reduction of the particle size, obtained for example using micronization techniques, leads to an increase in surface area and subsequently, according to the Noyes-Whitney equation, to an increase in the dissolution rate (Donald Lee Wise et al., Handbook of Pharmaceutical Controlled Release Technology, CRC Press, 2000, page 345). Therefore these techniques are commonly used when the drug substance has low solubility (Drug Bioavailability, Wiley-VCH, volume 40, page 545). On the other hand, when the drug substance has high solubility, such as metoprolol succinate, the techniques used to formulate it as an extended release pharmaceutical composition are those which reduce the solubility, as for example increasing the particle size or using another less soluble pharmaceutically acceptable salt.

Unexpectedly there are other advantages associated with the present invention. The process is more robust, increasing the reproducibility of the dissolution profile. See for example the reproducibility of the dissolution profile of examples 1 and 2, compared with that of examples 9 and 10. Another improvement was the increase of the metoprolol succinate content of the products (“Assay” according to European Pharmacopeia). This is an important parameter, which according to the Pharmacopeia should be between 90 and 110% for the final dosage form. This means that the assay of the granule must be more restrictive to ensure compliance (normally 95-105%). Using metoprolol succinate having a particle size bigger than that of the present invention the final assay was between 94 and 96%. In contrast the assay of the products of the present invention increases considerably being close to 100%.

For example, when reproducing the teachings of WO2008012346-A, the inventors were repeatedly faced with the fact that the dissolution profiles, obtained with the same batches of the starting API, metoprolol succinate, were quite different. This meant that many of the industrial batches had to be discarded because they were out of specifications (for instance a dissolution of more than 60% of metoprolol succinate at 8 h), making the process unprofitable from an economical point of view.

It was not until the invention was performed by using the technical features of claim 1 that batches were produced with reproducible dissolution profiles, lowering significantly the variability of dissolution profiles obtained from a single batch of metopropol succinate. Before this, many tests were performed varying parameters such as:

the proportion of excipients

the concentration of extended release agent in the coating solution

the % of coating

the temperature and time of the granulation and coating processes

the spray rates of coating agent

the spray gun nozzle diameter

the type of spray cap for spray gun

the distribution of particle size distribution of the granulate

without achieving a reproducible process in terms of dissolution profile and assay (content of active ingredient). More than 100 tests, pilot trials or pilot batches were performed to find a reproducible process.

Satisfactory clinical data had already been obtained with the composition of WO2008012346-A years ago, but the inventors were unable to scale-up a robust process to consistently obtain similar results. Dissolution profiles serve to somehow warrant that a given composition will have the same pharmacokinetic profile in humans as a reference medication that has been the subject of clinical studies.

The second aspect of the present invention relates to a granule comprising metoprolol succinate and at least two pharmaceutically acceptable excipients, wherein one pharmaceutically acceptable excipient is an extended release agent; the second pharmaceutically acceptable excipient is selected from a binder, a diluent and mixtures thereof; and metoprolol succinate is in a crystalline form having a D50 ranging from 5 to 16 microns and a D90 below 50 microns. Surprisingly, it has been found that the same technical effect is observed in the granule, before using it to form the extended release tablet.

The third aspect of the present invention is a process for the manufacture of a granule as defined in the previous aspects comprising the steps of:
i) Granulating metoprolol succinate, or a mixture comprising metoprolol succinate and at least one acceptable excipient, by the addition of a binding agent
ii) Optionally sieving or milling the product obtained

The granulation process of the present invention improves the granulation already known in the art, as for example in reducing the granulation time and being easier to calibrate.

The granules obtained can be further mixed with at least one pharmaceutically acceptable excipient and compressed to form tablets, and preferably the tablets are coated.

### DEFINITIONS

As used herein, the term “granulation” refers to the process of agglomerating powder particles into larger agglomerates (i.e. granules) that contain the active pharmaceutical ingredient. The term “granulation” includes wet granulation, dry granulation and melt granulation techniques. The resulting granulated mixture may be further processed, for example, through an extrusion and/or spheronisation process, and/or into various final dosage forms, e.g., capsules, tablets, wafers, gels, lozenges, etc.

The term “wet granulation” refers to any process comprising the steps of addition of a liquid to powder starting materials, preferably kneading, and drying to yield a solid dosage form.

As used herein, the term “melt granulation” refers to the process that comprises the steps of:

(a) forming a mixture of an active ingredient with at least one granulation excipient;
(b) granulating the mixture at a temperature that is less than or about the melting point (or melting range) of the active ingredient and
(c) cooling the product,
(d) calibrating of the product obtained after cooling.

The term “dry granulation” refers to any process comprising the compaction or compression of the powder or the starting materials in order to obtain a compacted product, as for example slugs, which are further calibrated to obtain granules with a suitable particle size.

The granulation excipient or excipients can be present in an amount from about 1% to about 95% by weight of the composition. In one embodiment, the granulation excipient may be present in an amount from about 25% to about 80% by weight of the composition. The active ingredient may be present in an amount from about 5% to about 99% by weight of the composition. In one embodiment, the active ingredient may be present in an amount of about 20% to about 75%.

As used herein, the term “extrusion” is understood as meaning a manufacturing process which is used to process a material or a blend of more than one material through a defined orifice, thereby giving rise to a homogeneous dispersion which possesses an extremely high degree of dispersity (or even a “solid solution”). In particular, in pharmaceutical technology, extrusion comprises an optional blending of different materials if present and the processing of these through a mesh of a sieve or a die of a perforated plate, often including an increase in pressure. The term “melt extrusion” as used herein is understood as meaning a special form of extrusion, where the temperature of the extruded material is raised beyond the melting temperature at a given process pressure of at least one of the components of the processed material. As is well-known to a person skilled in the art, the higher the process pressure, the faster the process. Consequently, the pressure used in the method of the invention is as high as acceptably possible.

The term “spheronisation” refers to the process of forming spherical particles.

As used herein, the term “granule” is not limited to the agglomerate directly obtained after the granulation processes, but it also refers to the product obtained after the extrusion and spheronisation processes.

The term “binder” or “binding agent” refer to any substance or mixture that exerts a physicochemical attractive force between molecules, and hence may be used in the formulation of a dosage form. In one embodiment of the invention, the binder or the binding agent may be mixed with other components of the composition, so that it is distributed uniformly throughout the dosage form. In one embodiment the binding agent is formed by the dissolution or dispersion of a binder in a liquid to form a binding solution or dispersion, and preferably the liquid is water. In another embodiment the binder is water. The binder may also provide a matrix upon which any additional components can associate. Binders include, but are not limited to, gelatin, polyvinylpyrrolidone (PVP), hydroxypropyl methylcellulose (HPMC), starch grades (pregelatinized or plain), hydroxypropylcellulose (HPC), and carboxymethylcellulose (CMC).

The term “diluent,” as used herein, refers to an agent or mixture of agents that when added to a formulation makes that formulation thinner or less concentrated and may also improve manufacturability. Diluents can be used to stabilize compounds because they can provide a more stable environment. In certain embodiments, diluents increase the bulk of the composition to facilitate compression or create sufficient bulk for a homogenous blend for capsule filling. Such compounds include e.g., lactose, starch, mannitol, sorbitol, dextrose, microcrystalline cellulose such as Avicel®, dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar), hydroxypropylmethylcellulose, hydroxypropylmethylcellulose acetate, sucrose-based diluents, confectioner's sugar; monobasic calcium sulfate monohydrate, calcium sulfate dihydrate; calcium lactate trihydrate, dextrates; hydrolyzed cereal solids, amylosem, powdered cellulose, calcium carbonate; glycine, kaolin, sodium chloride; inositol, bentonite, and the like.

The term “extended-release” is to be understood as defined in the United States Pharmacopeia 26, under the General Information section: “extended-release tablets are formulated in such manner as to make the continued medicament available over an extended period of time following ingestion”. Extended release is achieved by a special formulation design and/or manufacturing method. The specification for an extended release pharmaceutical composition comprising metoprolol succinate is disclosed in USP 32 (NF27 Vol. 3).

The term “extended release agent” refers to pharmaceutically acceptable excipients, or mixtures thereof, which delay the release of the active substance. These can be hydrophobic materials, and are often hydrocarbons and their derivatives such as lipids, waxes, paraffins and hydrophobic polymers; hydrophilic materials such as cellulose derivatives or methacrylic polymers; and inert non-erodible materials. Examples of “extended release agents” suitable for use herein include poorly water-soluble materials, such as, hydrocolloids, for example, alcohol-soluble cellulose derivatives such as ethyl cellulose and hydroxy-propyl cellulose; water soluble materials such as sodium alginate, pectin, gelatin, carrageenin, arabic acid, agar and karaya; and water insoluble waxes, such as carnauba wax, beeswax and microcrystalline waxes, polyvinyl alcohol, low molecular weight polyethylene, polyvinyl propionate. Preferred are ethylcellulose, sodium alginate and paraffin wax.

As used herein, the term “micronization” refers to a decrease in particle size through application of force to a particle, resulting in the break-up of the particle. Such force may be applied by collision of particles at high speeds.

The dissolution test was carried out according to the European Pharmacopeia 2.9.3. The HPLC detector was set up at λ=280 nm.

D50 and D90 represent the median or the 50th percentile and the 90th percentile of the particle size distribution, respectively, as measured by volume. That is, D50 (D90) is a value on the distribution such that 50% (90%) of the particles have a volume of this value or less.

The particle size distribution was measured using the following method:

### Equipment:

Beckman Coulter LS13320, module ULM. Measurement range: 0.04 to 2000 microns

### Fluid: Silicone oil

Optical model: Fraunhofer PIDS included

### Preparation of Sample:

Add 2 to 3 micro-spatulas of sample to a beaker containing 30-40 ml of silicone oil.

Re-suspend the sample using a plastic Pasteur pipette, aspirating several times.

Stir the sample in a magnetic stirrer for 10 minutes in order to break down the largest lumps.

Sonicate for 3 minutes (30 KHz, 200 W).

Place the sample in the analyser

Leave the sample to re-circulate in the apparatus for 15 minutes.

The sample is now ready for testing in triplicate

### Analyser Parameters:

Pump speed: 60%

Optical model: Fraunhofer PIDS included

Measuring time: 60 seconds

Number of measurements: 3

### Determination Method:

Set the parameters for the analysis of metoprolol succinate:

Obscuration required: 7%

Graph: frequency by channel and accumulated

Interpolation points in percentage: 10, 25, 50, 75 and 95

Interpolation points in micron: 1, 10, 50, 100 and 1000

Start test

Enter sample identification

Print out results

NB: Obscuration should be between 4 and 8%

### DETAILED DESCRIPTION OF THE INVENTION

In one embodiment metoprolol succinate has a D90 below 40 microns. Preferably metoprolol succinate has a D50 ranging from 7 to 12 microns.

Although the desired particle size can be obtained by several methods (e.g. by controlling the crystallization conditions), it has been observed that better results are obtained when the particle size is obtained by micronization.

How to manufacture extended release pharmaceutical compositions is well known. However, excellent results are obtained when the process comprises the step of granulating metoprolol succinate according to the present invention. The extended release agent can be added in several ways. One way would be by coating the granules with an extended release agent. Another would be by the adding the extended release agent during the granulation step. The granulation technique is not limited to wet granulation, but also includes melt and dry granulation. The granule can be used directly after calibrating, to reduce the particle size, and/or can be further processed. An alternative is that the granulation mixture is further extruded and spheronized.

The following examples are set out so as to provide those with ordinary skill in the art with a complete disclosure of how the products claimed herein are prepared, and are intended to be purely exemplary of the invention and are not intended to limit the scope of that which the inventors regard as their invention. Efforts have been made to ensure accuracy with respect to numbers (e.g., amounts, temperature, etc.), but some errors and deviations should be accounted for. Unless indicated otherwise, percentages are by weight, parts are parts by weight, temperature is in ° C. or is at room temperature, and pressure is at or near atmospheric pressure.

### EXAMPLES

### Preparation of an Extended Release Pharmaceutical Composition

### Example A

| COMPONENT | 190 mg tablet |
|---|---|
| Metoprolol succinate | 190.00 mg |
| Microcrystalline cellulose PH 101 | 591.20 mg |
| Methylcellulose 15 mPa · s | 95.00 mg |
| Glycerol | 1.90 mg |
| Maize starch | 15.50 mg |
| Purified water (*) | — |
| Ethylcellulose 100 mPa · s | 91.40 mg |
| Magnesium stearate | 15.00 mg |
| Isopropanol (*) | — |
| Acetone (*) | — |
| Sub-total | 1000.00 mg |
| Sepifilm ® LP 770 White | 30.00 mg |
| Total | 1030.00 mg |

(*) Mainly removed in the process.

Sepifilm® LP 770 White made up of approx: hypromellose 6/15 mPa·s (60.00-70.00%), microcrystalline cellulose 20 μm (5.00-15.00%), stearic acid (8.00-12.00%), titanium dioxide (E-171) (10.00-20.00%), purified water (*) 30.00 mg

### Method of Preparation:

Batch size: 190 kg of extended release pharmaceutical composition

### Machinery Used:

### Granulation:

Sieve

Stainless steel vessel with heating jacket and fitted with stirrer

Blender/Kneader

Wet granulator with 5 mm mesh screen

Fluid bed drier

Oscillating sieve

Vibrating sieve

### Coating of the Granulate:

Stainless steel vessel

Pneumatic propeller stirrer

Sieve

Pneumatic transfer pump

Airtight vessel with slow stirrer

Fluid bed equipment

Final Blending and Compressing of the Granulate into Cores:

Stainless steel sieves

Conical blender

Rotary tablet press

Tablet de-duster

### Coating of the Cores:

Vessel with stirrer

0.1 mm filter

Coating equipment

### Manufacturing Process:

### Granulation:

1. Check the weights of the starting materials.
2. Check the cleanliness of the manufacturing area.
3. Check that all material and equipment are clean and dry.
4. Sieve metoprolol succinate, microcrystalline cellulose PH101, methylcellulose maize starch and glycerol through a mesh screen.
5. Load the blender/kneader with the screened starting materials and blend for several minutes with the paddles.
6. Prepare the binding solution as follows:
   Place purified water into a stainless steel reactor fitted with a heating jacket and stirrer.
   Start the stirring and add the maize starch and glycerol. Stir until complete dispersion.
   Continue stirring and heat to 75-90° C.
   Once this temperature is reached and with constant stirring, cool the dispersion to room temperature.
   Once an homogenous, lump-free paste is obtained, dilute the paste to weight with purified water.
   Use immediately after preparation.
7. Transfer this maize starch paste to the blender/kneader. Knead for several minutes at an impeller speed of 100-300 rpm until a homogenous mixture with a suitable consistency for granulation is obtained.
8. Screen the mixture through a granulator fitted with a mesh screen.
9. Load the granulate into the fluid bed drier. Dry at an inlet air temperature of 45-60° C. until a residual water content of less than 4.0% is obtained, determined at 100-110° C. until constant weight. The approximate drying time is 1-3 hours.
10. Screen the dry granulate through an oscillating sieve.
11. Collect the granulate in a duly labelled airtight container.

### Coating of the Granulate

1. In a suitable stainless steel vessel containing isopropanol and acetone, add ethylcellulose N-100 and dissolve by pneumatic stirring. Stir until complete dissolution.
2. By means of a pneumatic pump sieve the solution though a mesh screen and collect in a suitable airtight container fitted with slow pneumatic stirrers (3-8 rpm).
3. Store the solution in an airtight container until the following day.
4. To compensate for any loss through evaporation of the solvents, if necessary make up the solution with isopropanol:acetone. Stir for several minutes at 15-30 rpm. Continue stirring at 3-15 rpm during the coating process
5. Load the granulate in the fluid bed equipment. Coat the granulate until a 26% increase in the theoretical weight is obtained. This percentage has been increased in two examples to evaluate its impact on the dissolution profile.
6. Coating parameters:
   Inlet air temperature: 30-50° C.
   Exhaust air temperature: 25-40° C.
   Solution flow rate: 450-550 g/min
   Inlet air flow rate: 1800-2500 m3/h
7. Collect the dry coated granulate in a suitable stainless steel container and weigh it.
8. Sieve microcrystalline cellulose PH101, magnesium stearate through a mesh screen. Sieve the coated granulate through a mesh screen.
9. Place the coated granulate, and the microcrystalline cellulose PH101 in the blender and blend for 5-15 minutes at 6 rpm.
10. Add the magnesium stearate. Blend for 4-10 minutes at 5-10 rpm. Weigh the final blend.

### Compression

1. Place the final mixture into the rotary tablet press hopper.
2. Adjust the parameters. Use oval, concave, 19×10 mm, punches scored on both sides. Compress the granulate into cores.
3. Determine the yield.
4. Take samples for Quality Control.

### Coating of the Cores

1. Preparation of coating suspension:
   Place purified water into a stainless steel container fitted with a stirrer. Slowly add Sepifilm.
   Stir for at least 45 minutes, until complete dispersion.
   Filter the suspension through a mesh screen.
   Continue stirring during the application of the coating.
2. Place the cores in the coating pan
3. Heat the cores to a temperature of 30-45° C., before starting the coating process
4. Coating process parameters are:
   Pan speed: 5-10 rpm
   Inlet air temperature: 50-65° C.
   Exhaust air temperature: 48-62° C.
   Spray pressure: 3 bar
5. Stop the process when the coated tablets have reached a weight increase of approx. 1.5% with respect to the cores.
6. Dry the coated tablets in the coating pan with intermittent turns until room temperature is reached.
7. Check the final weight of the coated tablets and calculate the yield.

### Results

The above procedure was repeated with several batches of metoprolol succinate each having different a particle size distribution.

The dissolution profile of the final pharmaceutical composition is given below:

| | Particle size distribution API (in microns) | | | Dissolution value (%) | | |
|---|---|---|---|---|---|---|
| | D 10 | D 50 | D 90 | 8 h | 20 h | Comments |
| Ex. 1 | 1.6 | 7.8 | 34.9 | 49.0 | 78.5 | Micronized API |
| Ex. 2 | 1.6 | 7.8 | 34.9 | 47.9 | 78.4 | Micronized API |
| Ex. 3 | 2.1 | 9.3 | 26.4 | 57.0 | 90.0 | Non-micronized API |
| Ex. 4 | 2.1 | 9.9 | 26.4 | 54.0 | 88.0 | Non-micronized API |
| Ex. 5 | 1.9 | 9.9 | 28.3 | 58.0 | 88.0 | Non-micronized API |
| Ex. 6 | 2.6 | 11.0 | 36.2 | 55.2 | 87.4 | Micronized API |
| Comparative | | | | | | |
| examples | | | | | | |
| Ex. 7 | 3.0 | 16.2 | 74.8 | 59.0 | 92.0 | |
| Ex. 8 | 3.5 | 17.7 | 68.7 | 60.0 | 92.0 | |
| Ex. 9 | 3.9 | 17.8 | 73.8 | 63.0 | 94.0 | |
| Ex. 10 | 3.9 | 17.8 | 73.8 | 52.0 | 85.0 | |
| Ex. 11 | 3.6 | 18.7 | 67.0 | 67.0 | 98.0 | 26.5% ethylcellulose in |
| | | | | | | the final weight of the |
| | | | | | | granule |
| Ex. 12 | 3.7 | 18.8 | 67.1 | 64.0 | 95.0 | 27.0% ethylcellulose in |
| | | | | | | the final weight of the |
| | | | | | | granule |
| Ex. 13 | 5.1 | 20.7 | 42.3 | 62.6 | 87.0 | |
| Ex. 13b | 4.2 | 21.9 | 76.3 | 60.0 | 91.0 | |
| Ex. 14 | 6.0 | 31.0 | 97.8 | 63.0 | 93.0 | |

The assay of the granule is given below:

| | D10 | D50 | D90 | % Assay |
|---|---|---|---|---|
| Ex. 15 micronized | 1.9 | 7.9 | 28.7 | 99.8 |
| Ex. 16 micronized | 1.9 | 7.9 | 28.7 | 97.0 |
| Ex. 17 non micronized | 2.9 | 19.1 | 84.2 | 95.3 |
| Ex. 18 non-micronized | 2.9 | 19.1 | 84.2 | 94.4 |

Satisfactory clinical data had already been obtained with the composition of WO2008012346-A years ago, but the inventors were unable to scale-up a robust process to consistently obtain similar results. Dissolution profiles serve to somehow warrant that a given composition will have the same pharmacokinetic profile in humans as a reference medication that has been the subject of clinical studies.

We provide additional examples of such lack or reproducibility here. Examples included as Table 1 and Table 2 (Table 1 providing additional data on the dissolution profile and assay of the compositions according to the invention compared with other compositions, and Table 2 providing additional data on the dissolution profile of the compositions according to the invention compared with other compositions) were prepared according to Example A of this application at two different manufacturing sites. Tests 1-22 and 35-48 were performed according to the invention, while test 23-34 and 49-75 were comparative tests. In tests according to the invention, batches 1-5 and 8 of metoprolol succinate (API) had features according to claim 1; while in comparative tests, batches 6-7 and 9, the active was out of the scope of claim 1.

It is surprising that the standard deviation of the % dissolved at 8 h and 20 h is much lower when starting from the same API batch in the batches manufactured according to the invention (at 8 h 1.0, 1.3, 0.9, 2.6, 2.6 and 1.8, for tests 1-4, 5-8, 9-12, 13-18, 19-22 and 35-48 according to the invention; compared with 6.9, 4.9 and 5.0 for tests 23-27, 28-34 and 49-75 which are comparative tests; and at 20 h standard deviations of 2.1, 0.8, 1.6, 1.6, 1.2 and 2,1 were found in the tests according to the invention, compared with 4.5, 3.0 and 4.2 for tests 23-27, 28-34 and 49-75 which are comparative tests).

It is also surprising that tablets which include API (metoprolol succinate) with a smaller particle size according to the invention such as tests 1-22, have means of % of dissolved metoprolol succinate at 8 h (clearly below 60%) lower than tablets of comparative tests 23-34 (which are above 60%). The same occurs with tests 35-48 in Table 2 according to the invention (having a mean of 54.1%), compared to tests 49-75 which have a mean of % dissolved at 8 h of 62.7%; i.e. it is surprising that the extended release effect is more pronounced with the use of an API with a smaller particle size. The extended release effect is even more pronounced when the API is micronized.

Additionally the final content (assay) of metoprolol succinate found in the tablets was increased improving the compliance of the requirements of the European Pharmacopeia.

TABLE 1

| | | | | | | Micronized/ | According | | | | |
|---|---|---|---|---|---|---|---|---|---|---|---|
| | | | | | | Non- | to the | Dissolution Profile | | | |
| Test | strength | API | PSD | | | micronized | inven- | % of metoprolol succinate dissolved | | | |
| # | (mg) | batch | d 10 | d 50 | d 90 | API? | tion? | 1 h: <25% | 4 h: 20-40% | 8 h: 40-60% | 20 h >80% |
| 1 | 23.75 | 1 | 2.122 | 9.867 | 28.270 | Non | [symbol] | 13 | 33 | 54 | 88 |
| 2 | 47.50 | | 2.122 | 9.867 | 28.270 | micronized | | 13 | 35 | 56 | 92 |
| 3 | 95.00 | | 2.122 | 9.867 | 28.270 | small parti- | | 13 | 34 | 56 | 87 |
| 4 | 190.00 | | 2.122 | 9.867 | 28.270 | cle size | | 13 | 34 | 56 | 87 |
| 5 | 23.75 | 2 | 1.870 | 9.908 | 28.250 | | [symbol] | 14 | 37 | 58 | 88 |
| 6 | 47.50 | | 1.870 | 9.908 | 28.250 | | | 13 | 34 | 57 | 87 |
| 7 | 95.00 | | 1.870 | 9.908 | 28.250 | | | 13 | 35 | 56 | 87 |
| 8 | 190.00 | | 1.870 | 9.908 | 28.250 | | | 12 | 34 | 55 | 86 |
| 9 | 23.75 | 3 | 2.680 | 11.890 | 34.180 | | [symbol] | 15 | 35 | 57 | 90 |
| 10 | 47.50 | | 2.680 | 11.890 | 34.180 | | | 13 | 33 | 57 | 90 |
| 11 | 95.00 | | 2.680 | 11.890 | 34.180 | | | 14 | 34 | 57 | 88 |
| 12 | 190.00 | | 2.680 | 11.890 | 34.180 | | | 12 | 32 | 55 | 87 |
| 13 | 47.50 | 4 | 2.632 | 11.03 | 36.21 | Micronized | [symbol] | 9 | 27 | 49 | 83 |
| 14 | 23.75 | | 2.632 | 11.03 | 36.21 | API | | 7 | 28 | 51 | 86 |
| 15 | 47.50 | | 2.632 | 11.03 | 36.21 | | | 10 | 31 | 55 | 87 |
| 16 | 47.50 | | 2.632 | 11.03 | 36.21 | | | 10 | 31 | 55 | 87 |
| 17 | 47.50 | | 2.632 | 11.03 | 36.21 | | | 9 | 27 | 50 | 84 |
| 18 | 47.50 | | 2.632 | 11.03 | 36.21 | | | 9 | 29 | 53 | 85 |
| 19 | 47.50 | 5 | 1.600 | 7.800 | 34.900 | | [symbol] | 8 | 26 | 49 | 84 |
| 20 | 95.00 | | 1.600 | 7.800 | 34.900 | | | 9 | 26 | 47 | 82 |
| 21 | 95.00 | | 1.600 | 7.800 | 34.900 | | | 8 | 23 | 45 | 82 |
| 22 | 95.00 | | 1.600 | 7.800 | 34.900 | | | 9 | 28 | 51 | 84 |
| Comparative | | | | | | | | | | | |
| examples | | | | | | | | | | | |
| 23 | 95.00 | 6 | 3.313 | 23.42 | 53.33 | Non | [symbol] | 13 | 41 | 65 | 93 |
| 24 | 190.00 | | 3.313 | 23.42 | 53.33 | micronized | | 14 | 41 | 65 | 90 |
| 25 | 95.00 | | 3.313 | 23.42 | 53.33 | big particle | | 14 | 47 | 76 | 99 |
| 26 | 95.00 | | 3.313 | 23.42 | 53.33 | size | | 16 | 52 | 76 | 99 |
| 27 | 47.50 | | 3.313 | 23.42 | 53.33 | | | 13 | 39 | 61 | 86 |
| 28 | 190.00 | 7 | 5.060 | 20.720 | 42.300 | | [symbol] | 12 | 35 | 58 | 88 |
| 29 | 190.00 | | 5.060 | 20.720 | 42.300 | | | 12 | 33 | 55 | 86 |
| 30 | 47.50 | | 5.060 | 20.720 | 42.300 | | | 11 | 46 | 68 | 85 |
| 31 | 95.00 | | 5.060 | 20.720 | 42.300 | | | 11 | 35 | 61 | 87 |
| 32 | 47.50 | | 5.060 | 20.720 | 42.300 | | | 13 | 40 | 64 | 91 |
| 33 | 47.50 | | 5.066 | 20.720 | 42.330 | | | 13 | 38 | 62 | 91 |
| 34 | 47.50 | | 5.066 | 20.720 | 42.330 | | | 15 | 44 | 68 | 93 |

| | | | | | Average of | | | |
|---|---|---|---|---|---|---|---|---|
| Test | STDV | STDV | Mean 8 h % | Mean 20 h % | means | | | Average of |
| # | 8 h | 20 h | dissolved | dissolved | 8 h | 20 h | Assay % | assay values |
| 1 | 1.0 | 2.1 | 55.6 | 88.4 | 56.1 | 88.0 | 99.2 | 99.7 |
| 2 | | | | | | | 99.0 | |
| 3 | | | | | | | 103.2 | |
| 4 | | | | | | | 101.3 | |
| 5 | 1.3 | 0.8 | 56.5 | 86.9 | | | 102.3 | |
| 6 | | | | | | | 96.4 | |
| 7 | | | | | | | 96.3 | |
| 8 | | | | | | | 97.8 | |
| 9 | 0.9 | 1.6 | 56.4 | 88.8 | | | 96.8 | |
| 10 | | | | | | | — | |
| 11 | | | | | | | 103.3 | |
| 12 | | | | | | | 100.7 | |
| 13 | 2.6 | 1.6 | 52.2 | 85.3 | 50.1 | 84.2 | 101.1 | 99.5 |
| 14 | | | | | | | 99.8 | |
| 15 | | | | | | | 100.1 | |
| 16 | | | | | | | 97.7 | |
| 17 | | | | | | | 100.0 | |
| 18 | | | | | | | 99.8 | |
| 19 | 2.6 | 1.2 | 48.0 | 83.0 | | | 100.0 | |
| 20 | | | | | | | 100.2 | |
| 21 | | | | | | | 99.3 | |
| 22 | | | | | | | 97.3 | |
| Comparative | | | | | | | | |
| examples | | | | | | | | |
| 23 | 6.9 | 4.5 | 68.6 | 93.4 | 65.4 | 91.1 | [symbol] | 95.1 |
| 24 | | | | | | | [symbol] | |
| 25 | | | | | | | 96.7 | |
| 26 | | | | | | | 95.7 | |
| 27 | | | | | | | 94.4 | |
| 28 | 4.9 | 3.0 | 62.3 | 88.7 | | | 95.1 | |
| 29 | | | | | | | 96.0 | |
| 30 | | | | | | | [symbol] | |
| 31 | | | | | | | 93.1 | |
| 32 | | | | | | | 94.0 | |
| 33 | | | | | | | 95.8 | |
| 34 | | | | | | | 95.5 | |

TABLE 2

| | | | | | | Micronized/ | | Dissolution profile | | | | | | | |
|---|---|---|---|---|---|---|---|---|---|---|---|---|---|---|---|
| | | | | | | Non- | According | % of metoprolol succinate dissolved | | | | | | | |
| Test | | API | PSD | | | micronized | to the | 1 h | 4 h | 8 h | 20 h | STD | STD | Mean | Mean |
| # | strenght | Batch | d 10 | d 50 | d 90 | API? | invention? | (<25%) | (20-40%) | (40-60%) | (>80%) | 8 h | 20 h | 8 h | 20 h |
| 35 | 47.5 | 8 | 1.8 | 8 | 31.4 | Micro- | [symbol] | 11 | 33 | 54 | 84 | 1.8 | 2.1 | 54.1 | 83.4 |
| 36 | 47.5 | | 1.8 | 8 | 31.4 | nized | | 12 | 32 | 51 | 80 | | | | |
| 37 | 95.0 | | 1.8 | 8 | 31.4 | | | 11 | 33 | 51 | 83 | | | | |
| 38 | 47.5 | | 1.8 | 8 | 31.4 | | | 12 | 32 | 52 | 82 | | | | |
| 39 | 190.0 | | 1.8 | 8 | 31.4 | | | 12 | 33 | 54 | 85 | | | | |
| 40 | 47.5 | | 1.8 | 8 | 31.4 | | | 12 | 33 | 53 | 82 | | | | |
| 41 | 47.5 | | 1.8 | 8 | 31.4 | | | 12 | 35 | 56 | 82 | | | | |
| 42 | 47.5 | | 1.8 | 8 | 31.4 | | | 12 | 35 | 56 | 82 | | | | |
| 43 | 47.5 | | 1.8 | 8 | 31.4 | | | 12 | 33 | 54 | 83 | | | | |
| 44 | 190.0 | | 1.8 | 8 | 31.4 | | | 13 | 34 | 55 | 86 | | | | |
| 45 | 47.5 | | 1.8 | 8 | 31.4 | | | 11 | 33 | 55 | 88 | | | | |
| 46 | 190.0 | | 1.8 | 8 | 31.4 | | | 12 | 34 | 57 | 85 | | | | |
| 47 | 95.0 | | 1.8 | 8 | 31.4 | | | 11 | 33 | 55 | 84 | | | | |
| 48 | 190.0 | | 1.8 | 8 | 31.4 | | | 12 | 32 | 54 | 81 | | | | |
| Comparative | | | | | | | | | | | | | | | |
| examples | | | | | | | | | | | | | | | |
| 49 | 47.5 | 9 | 3.5 | 17.7 | 68.7 | Non- | [symbol] | 15 | 39 | 63 | 95 | 5.0 | 4.2 | 62.7 | 93.7 |
| 50 | 47.5 | | 3.5 | 17.7 | 68.7 | micro- | | 16 | 40 | 63 | 94 | | | | |
| 51 | 47.5 | | 3.5 | 17.7 | 68.7 | nized | | 14 | 36 | 59 | 92 | | | | |
| 52 | 95.0 | | 3.5 | 17.7 | 68.7 | | | 16 | 40 | 63 | 93 | | | | |
| 53 | 95.0 | | 3.5 | 17.7 | 68.7 | | | 14 | 37 | 60 | 92 | | | | |
| 54 | 95.0 | | 3.5 | 17.7 | 68.7 | | | 13 | 32 | 52 | 85 | | | | |
| 55 | 190.0 | | 3.5 | 17.7 | 68.7 | | | 13 | 31 | 51 | 84 | | | | |
| 56 | 190.0 | | 3.5 | 17.7 | 68.7 | | | 16 | 39 | 61 | 90 | | | | |
| 57 | 190.0 | | 3.5 | 17.7 | 68.7 | | | 13 | 33 | 54 | 86 | | | | |
| 58 | 23.75 | | 3.5 | 17.7 | 68.7 | | | 21 | 44 | 66 | 97 | | | | |
| 59 | 47.5 | | 3.5 | 17.7 | 68.7 | | | 14 | 38 | 61 | 94 | | | | |
| 60 | 95.0 | | 3.5 | 17.7 | 68.7 | | | 15 | 36 | 59 | 92 | | | | |
| 61 | 47.5 | | 3.5 | 17.7 | 68.7 | | | 17 | 40 | 70 | 102 | | | | |
| 62 | 47.5 | | 3.5 | 17.7 | 68.7 | | | 17 | 40 | 63 | 93 | | | | |
| 63 | 47.5 | | 3.5 | 17.7 | 68.7 | | | 17 | 41 | 65 | 96 | | | | |
| 64 | 47.5 | | 3.5 | 17.7 | 68.7 | | | 15 | 38 | 63 | 95 | | | | |
| 65 | 47.5 | | 3.5 | 17.7 | 68.7 | | | 15 | 39 | 64 | 95 | | | | |
| 66 | 47.5 | | 3.5 | 17.7 | 68.7 | | | 16 | 46 | 72 | 101 | | | | |
| 67 | 47.5 | | 3.5 | 17.7 | 68.7 | | | 16 | 42 | 67 | 97 | | | | |
| 68 | 47.5 | | 3.5 | 17.7 | 68.7 | | | 13 | 39 | 64 | 93 | | | | |
| 69 | 190.0 | | 3.5 | 17.7 | 68.7 | | | 16 | 40 | 62 | 94 | | | | |
| 70 | 190.0 | | 3.5 | 17.7 | 68.7 | | | 16 | 40 | 63 | 94 | | | | |
| 71 | 190.0 | | 3.5 | 17.7 | 68.7 | | | 16 | 41 | 67 | 98 | | | | |
| 72 | 190.0 | | 3.5 | 17.7 | 68.7 | | | 16 | 40 | 66 | 95 | | | | |
| 73 | 190.0 | | 3.5 | 17.7 | 68.7 | | | 15 | 37 | 59 | 90 | | | | |
| 74 | 190.0 | | 3.5 | 17.7 | 68.7 | | | 16 | 42 | 68 | 98 | | | | |
| 75 | 190.0 | | 3.5 | 17.7 | 68.7 | | | 16 | 42 | 68 | 96 | | | | |

1. An extended release pharmaceutical composition comprising metoprolol succinate and at least two pharmaceutically acceptable excipients, wherein one pharmaceutically acceptable excipient is an extended release agent; the second pharmaceutically acceptable excipient is selected from a binder, a diluent and mixtures thereof; and metoprolol succinate is in a crystalline form having a D50 ranging from 5 to 16 microns and a D90 below 50 microns.

2. The pharmaceutical composition according to claim 1, wherein metoprolol succinate has a D90 below 40 microns.

3. The pharmaceutical composition according to claim 2, wherein metoprolol succinate has a D50 ranging from 7 to 12 microns.

4. The pharmaceutical composition according to claim 1, wherein metoprolol succinate has been milled or micronized to reduce the particle size.

5. The pharmaceutical composition according to claim 4, wherein metoprolol succinate has been micronized to reduce the particle size.

6. The pharmaceutical composition according to claim 1 comprising granules comprising metoprolol succinate.

7. A granule comprising metoprolol succinate and at least two pharmaceutically acceptable excipients, wherein one pharmaceutically acceptable excipient is an extended release agent; the second pharmaceutically acceptable excipient is selected from a binder, a diluent and mixtures thereof; and metoprolol succinate is in a crystalline form having a D50 ranging from 5 to 16 microns and a D90 below 50 microns.

8. The granule according to claim 7, wherein metoprolol succinate has a D90 below 40 microns.

9. The granule according to claim 8, wherein metoprolol succinate has a D50 ranging from 7 to 12 microns.

10. The granule according to claim 7, wherein metoprolol succinate has been milled or micronized to reduce the particle size, and preferably it has been micronized.

11. The use of a granule as defined in claim 7, for the manufacture of an extended release pharmaceutical composition.

12. A process for the manufacture of a granule as defined in claim 7, comprising the steps of:
i) Granulating metoprolol succinate, or a mixture comprising metoprolol succinate and at least one acceptable excipient, by the addition of a binding agent
ii) Optionally sieving or milling the product obtained.

13. The process according to claim 12, further comprising the step of:
iii) Coating the product obtained in the step (ii) with an extended release agent, preferably ethylcellulose.

14. The process according to claim 12, wherein the granulation comprises an extended release agent.

15. The process according to claim 12, further comprising after step (i) the steps of:
i′) extruding the product obtained in the step (i)
i″) spheronizing the extruded product obtained in step (i′)

16. A process for the manufacture of an extended release pharmaceutical composition comprising the process steps as defined in claim 12, wherein the final granule or spheronized granule is further mixed with at least one pharmaceutically acceptable excipient and compressed to form a tablet, and preferably the tablet is coated.
